(19) United States
(12) Patent Application Publication
(10) Pub. No.: US 2024/0301296 A1
(43) Pub. Date: Sep. 12, 2024

(54) MULTISTAGE REACTOR SYSTEMS AND PROCESSES FOR CONVERSION OF OXYGENATES

(71) Applicant: Gevo, Inc., Englewood, CO (US)

(72) Inventors: Andrew J. Ingram, Denver, CO (US); Robert P. Hepfer, Saint Charles, IL (US); Brian O'Neill, Boulder, CO (US)

(21) Appl. No.: 18/599,073

(22) Filed: Mar. 7, 2024

Related U.S. Application Data
(60) Provisional application No. 63/488,867, filed on Mar. 7, 2023.

(51) Int. Cl. C10G 3/00

(52) U.S. Cl. CPC C10G 3/40; C10G 3/49; C10G 3/60; C10G 2300/4006; C10G 2400/20

(57) ABSTRACT

Exemplary processes for converting one or more oxygenates to one or more olefins are disclosed. In one exemplary aspect, the process can include introducing a first input feed into an adiabatic multistage reactor with a first reactor bed and a second reactor bed. The process can also include contacting the first input feed with the first reactor bed to maintain a first temperature of the first reactor bed within a first temperature range and to produce a first reaction mixture, and introducing a second input feed into the multistage reactor such that the first reaction mixture is mixed with the second input feed to produce a first effluent. The process can also include contacting the first effluent with the second reactor bed to thereby maintain a second temperature of the second reactor bed within a second temperature range and to produce a second reaction mixture. Exemplary systems are also disclosed.

### CROSS-REFERENCE TO RELATED APPLICATION

This application claims priority to U.S. Provisional Application No. 63/488,867 filed Mar. 7, 2023, the entire contents of which are hereby expressly incorporated by reference herein.

### TECHNICAL FIELD

Multistage reactor systems and processes for conversion of oxygenates (e.g., alcohols or ethers), and more specifically, adiabatic processes resulting in the conversion of oxygenates to olefins are provided.

### BACKGROUND

Multistage reactor systems and processes for conversion of oxygenates (e.g., alcohols or ethers), and more specifically, to adiabatic processes resulting in the conversion of oxygenates to olefins are provided.

Fixed bed adiabatic reactors are among the least expensive reactors available, but present a challenge for processes that release or absorb significant amounts of heat. Endothermic reactions reduce the temperature of the process as a function of extent of reaction and can quench processes without adding heat. Exothermic reactions increase the temperature of the process as a function of extent of reaction and can cause the reaction to lose selectivity or proceed uncontrollably without removing heat. Adding or removing heat in such processes would require expensive heat exchange equipment, ultimately increase net heat demand, increase system pressure drop, and increase the difficulty to control the process. In processes with high heat exchange demand, it is typical to segment a fixed bed and add heating or cooling stages, or use the generally more expensive multi-tubular fixed bed reactors where the reactor itself serves as a heat exchanger. However, such systems can be inefficient, and moreover, the applicability of such systems can be severely limited (e.g., steam methane reforming or partial oxidation of propylene to acrylic acid among others) and generally cannot handle systems that are a combination of exothermic and endothermic steps.

Accordingly, there remains a need for improved, efficient, cost effective, and more versatile systems and processes that involve and can combine endothermic and exothermic reactions.

### BRIEF DESCRIPTION OF THE DRAWINGS

The accompanying drawings, which are incorporated into and constitute a part of this specification, show certain aspects of the subject matter disclosed herein and, together with the description, help explain some of the principles associated with the disclosed implementations. In the drawings:

FIG. 1 is a schematic illustration of an exemplary system for conversion of oxygenates where the first input feed includes at least ethanol; and

FIG. 2 is a schematic illustration of another exemplary system for conversion oxygenates where the first input feed includes at least methanol and ethanol.

When practical, similar reference numbers denote similar structures, features, or elements.

### SUMMARY

In certain aspects of the current subject matter, challenges associated with conversion of oxygenates can be addressed by inclusion of one or more of the features described herein or comparable/equivalent approaches as would be understood by one of ordinary skill in the art. Aspects of the current subject matter relate to processes and systems for one or more oxygenates to one or more olefins.

In some aspects, one or more of the following features may optionally be included in any feasible combination.

Exemplary processes for converting one or more oxygenates to one or more olefins are disclosed. In one exemplary aspect, the process includes introducing a first input feed into a first end of an adiabatic multistage reactor, the first input feed including one or more first oxygenates and one or more first olefins, the multistage reactor having at least a first reaction stage and a second reaction stage. The first reaction stage is upstream of the second reaction stage, and the first reaction stage has a first reactor bed, and the second reaction stage has a second reactor bed. The process also includes contacting the first input feed with the first reactor bed to thereby maintain a first temperature of the first reactor bed within a first temperature range from about 300° C. to 550° C. and to produce a first reaction mixture, introducing a second input feed into the multistage reactor downstream of the first reaction stage such that, upon exiting the first reaction stage, the first reaction mixture is mixed with the second input feed to produce a first effluent having a different composition relative to the first reaction mixture, the second input feed including one or more second oxygenates. The process also includes contacting the first effluent with the second reactor bed to thereby maintain a second temperature of the second reactor bed within a second temperature range from about 300° C. to 550° C. and to produce a second reaction mixture.

In some aspects, the multistage reactor can have a third reaction stage, the third reaction stage having a third reactor bed. In this case, the process can further include introducing a third input feed into the multistage reactor downstream of the second reaction stage such that, upon exiting the second reaction stage, the second reaction mixture can be mixed with the third input feed to produce a second effluent having a different composition relative to the second reaction mixture, the third input feed including one or more third oxygenates. The process also includes contacting the second effluent with the third reactor bed to thereby maintain a third temperature of the third reactor bed within a third temperature range from about 300° C. to 550° C. and to produce a third reaction mixture.

In some aspects, during each reaction stage and between reaction stages, external heat is not added to the multistage reactor. In some aspects, during each reaction stage and between reaction stages, heat is not removed from the multistage reactor. In some aspects, the first effluent is not removed from the multistage reactor. In some aspects, the second effluent is not removed from the multistage reactor. In some aspects, heat is not removed from the first reaction mixture prior to being mixed with the second input feed. In some aspects, heat is not removed from the second reaction mixture prior to being removed from the multistage reactor or subsequently mixed with the third input feed. In some aspects, heat is not removed from the third reaction mixture prior to being removed from the multistage reactor or subsequently mixed with a fourth input feed.

In some aspects, the one or more first oxygenates include a predominant first oxygenate, and the one or more first olefins includes a predominant first olefin, wherein the predominant first oxygenate can be ethanol and the predominant first olefin can be ethylene. In some aspects, the molar ratio of ethylene:ethanol can be from about 0.25 to 10 in the first input feed. In some aspects, the molar ratio of ethylene:ethanol can be from about 0.25 to 5 in the first input feed. In some aspects, the first temperature range can be from about 350° C. to 500° C. In some aspects, the second temperature range can be from about 350° C. to 500° C. In some aspects, the third temperature range can be from about 350° C. to 500° C.

In some aspects, at least one of the first reactor bed or the second reactor bed can be a fixed bed. In some aspects, the third reactor bed can be a fixed bed. In some aspects, at least one of the first reactor bed or the second reactor bed can be a fluidized bed. In some aspects, the third reactor bed can be a fluidized bed. In some aspects, at least one of the first reactor bed or the second reactor bed can be a moving bed. In some aspects, the third reactor bed can be a moving bed.

In some aspects, the one or more first oxygenates and the one or more second oxygenates can be the same. In some aspects, the one or more first oxygenates, the one or more second oxygenates, and the one or more third oxygenates can be the same. In some aspects, the one or more first oxygenates include one or more C2+ alcohols. In some aspects, the one or more second oxygenates include one or more C2+ alcohols. In some aspects, the one or more third oxygenates include one or more C2+ alcohols.

In some aspects, the one or more first oxygenates include a predominate first oxygenate, wherein the predominant oxygenate can be ethanol. In some aspects, the one or more second oxygenates includes a second predominant oxygenate, wherein the predominant oxygenate can be ethanol. In some aspects, the one or more third oxygenates include a predominant third oxygenate, wherein the predominant oxygenate can be ethanol.

In some aspects, the process can further include introducing the second reaction mixture into a single stage reactor, the single stage reactor including one or more catalysts, and contacting the second reaction mixture with the one or more catalysts to produce an output stream including one or more product olefins. Prior to introducing the second reaction mixture into the single stage reactor, the process can include decreasing the temperature of the second reaction mixture.

In some aspects, the process can further include introducing the output stream into a separation subsystem to produce a first stream and a second stream.

In some aspects, the second stream can include at least one C3+ olefins. In some aspects, the process can further include combining the first stream with the one or more first oxygenates to produce the first input feed. In some aspects, the first stream can include a predominant olefin, wherein the predominant olefin can be ethylene.

In some aspects, the process can further include condensing the output stream into a condensed output stream and introducing the condensed output stream into a separation subsystem to produce a first stream and a second stream. In some aspects, the process can further include combining the first stream with the one or more first oxygenates to produce the first input feed. In some aspects, the first stream can include a predominant olefin, wherein the predominant olefin can be ethylene. In some aspects, the second stream can include at least one C3+ olefin.

In some aspects, the process can further include introducing the third reaction mixture into a single stage reactor, the single stage reactor including one or more catalysts, and contacting the third reaction mixture with the one or more catalysts to produce an output stream including one or more product olefins.

In some aspects, prior to introducing the third reaction mixture into the single stage reactor, the process can further include decreasing the temperature of the third reaction mixture. In some aspects, the process can further include introducing the output stream into a separation system to produce a first stream and a second stream. In some aspects, the process can further include combining the first stream with the one or more first oxygenates to produce the first input feed. In some aspects, the first stream can include a predominant olefin, wherein the predominant olefin can be ethylene. In some aspects, the second stream can include at least one C3+ olefin.

In some aspects, the process can further include condensing the output stream into a condensed output stream and introducing the condensed reaction mixture into a separation subsystem to produce a first stream and a second stream. In some aspects, the process can further include combining the first stream with the one or more first oxygenates to produce the first input feed. In some aspects, the first stream can include a predominant olefin, wherein the predominant olefin can be ethylene. In some aspects, the second stream can include at least one C3+ olefin.

In some aspects, prior to introducing the first input stream into the adiabatic multistage reactor, the process can further include superheating the one or more first oxygenates. In some aspects, prior to introducing the second input stream into the adiabatic multistage reactor, the process can further include superheating the one or more second oxygenates. In some aspects, prior to introducing the third input stream into the adiabatic multistage reactor, the process can further include superheating the one or more third oxygenates.

In some aspects, the multistage reactor can be at a gauge pressure from 0 to about 30 bar. In some aspects, the multistage reactor can be at a weight hourly space velocity (WHSV) from about 0.25 to 15.

In some aspects, at least one of the first reactor bed or the second reactor bed can include a mixture of catalysts. In some aspects, the mixture of catalysts can include a zeolite and an alcohol dehydration catalyst. In some aspects, the third reactor bed can include a mixture of catalysts. In some aspects, the mixture of catalysts of the third reactor bed can include a zeolite and an alcohol dehydration catalyst.

In some aspects, the second input feed can be introduced into the adiabatic multistage reactor at a temperature that can be greater than a temperature of the first reaction mixture. In some aspects, the third input feed can be introduced into the adiabatic multistage reactor at a temperature that can be greater than a temperature of the second reaction mixture.

In some aspects, the multistage reactor can include one or more additional reaction stages downstream of the third reaction stage, the one or more additional reaction stages each having a respective reactor bed. In this case the process can further include introducing a subsequent input feed into the multistage reactor downstream of prior reaction stages such that, upon exiting the prior reaction stage, a prior reaction mixture can be mixed with the subsequent input feed to produce an additional effluent having a different composition than the prior reaction mixture, the subsequent input feed including one or more additional oxygenates. The process can also include contacting the prior effluent with the respective reactor bed of one of the one of more additional reaction stages to thereby maintain a temperature of the respective reactor bed within a temperature range from about 300° C. to 550° C. and to produce an additional reaction mixture downstream of the prior reaction stages.

In some aspects, the one or more first oxygenates can include ethanol and methanol. In some aspects, the one or more first olefins can include ethylene, propylene, butene, or any combination thereof.

In some aspects, the one or more first oxygenates do not include methanol. In some aspects, the one or more second oxygenates do not include methanol. In some aspects, the one or more third oxygenates do not include methanol.

In another aspect, another process for converting one or more oxygenates to one or more olefins is provided. In some aspects, the process includes introducing a first input feed into a first end of an adiabatic multistage reactor, the first input feed includes one or more first oxygenates, the one or more first oxygenates include ethanol and at least one of methanol or dimethylether, the multistage reactor has at least a first reaction stage and a second reaction stage, wherein the first reaction stage is upstream of the second reaction stage, and the first reaction stage has a first reactor bed, and the second reaction stage has a second reactor bed. The process also includes contacting the first input feed with the first reactor bed to thereby maintain a first temperature of the first reactor bed within a first temperature range from about 300° C. to 550° C. and to produce a first reaction mixture and introducing a second input feed into the multistage reactor downstream of the first reaction stage such that, upon exiting the first reaction stage, the first reaction mixture is mixed with the second input feed to produce a first effluent having a different composition relative to the first reaction mixture, the second input feed includes one or more second oxygenates. The process further includes contacting the first effluent with the second reactor bed to thereby maintain a second temperature of the second reactor bed within a second temperature range from about 300° C. to 550° C. and to produce a second reaction mixture.

In some aspects, the multistage reactor can have a third reaction stage having a third reactor bed and the process can further include introducing a third input feed into the multistage reactor downstream of the second reaction stage such that, upon exiting the second reaction stage, the second reaction mixture can be mixed with the third input feed to produce a second effluent having a different composition relative to the second reaction mixture, the third input feed including one or more third oxygenates. The process can also include contacting the second effluent with the third reactor bed to thereby maintain a third temperature of the third reactor bed within a third temperature range from about 300° C. to 550° C. and to produce a third reaction mixture. In some aspects, the one or more first oxygenates can include ethanol and methanol. In some aspects, the one or more first oxygenates can include ethanol or dimethylether. In such aspects, the one or more first oxygenates can further include methanol.

In some aspects, the one or more second oxygenates can include ethanol, methanol, or a combination thereof. In some aspects, one or more second oxygenates do not include methanol.

In some aspects, the one or more third oxygenates can include ethanol, methanol, or a combination thereof. In some aspects, the one or more third oxygenates do not include methanol.

In another aspect, a system arranged to convert one or more oxygenates to one or more olefins using the processes described above is provided. In some aspects, the system can include the adiabatic multistage reactor described above.

In another aspect, a process for converting one or more oxygenates to one or more can include a first input feed into a first end of adiabatic multistage reactor assembly, the first input feed can include one or more first oxygenates and at least one or more first olefins and methanol, where the multistage reactor assembly has at least a first reaction stage and a second reaction stage, and the first reaction stage is upstream of the second reaction stage. The first reaction stage has a first reactor bed, and the second reaction stage has a second reactor bed. The process also includes contacting the first input feed with the first reactor bed to thereby maintain a first temperature of the first reactor bed within a first temperature range from about 300° C. to 550° C. and to produce a first reaction mixture, and introducing a second input feed into the multistage reactor assembly downstream of the first reaction stage such that, upon exiting the first reaction stage, the first reaction mixture is mixed with the second input feed to produce a first effluent having a different composition relative to the first reaction mixture. The second input feed includes one or more second oxygenates. The process also includes contacting the first effluent with the second reactor bed to thereby maintain a second temperature of the second reactor bed within a second temperature range from about 300° C. to 550° C. and to produce a second reaction mixture.

In some aspects, the multistage reactor assembly can have a third reaction stage, where the third reaction stage has a third reactor bed. In such aspects, the process can include introducing a third input feed into the multistage reactor assembly downstream of the second reaction stage such that, upon exiting the second reaction stage, the second reaction mixture can be mixed with the third input feed to produce a second effluent that can have a different composition relative to the second reaction mixture. The third input feed can include one or more third oxygenates. The process can also include contacting the second effluent with the third reactor bed to thereby maintain a third temperature of the third reactor bed within a third temperature range from about 300° C. to 550° C. and to produce a third reaction mixture.

In some aspects, the multistage reactor assembly can include two or more reactors, in which the first and second stages can be carried in the first reactor and the third stage can be carried out in the second reactor.

In some aspects, the multistage reactor assembly can include two or more reactors, in which the first reactor bed can be located in a first reactor and the second reactor bed can be located in a second reactor. In such aspects, the third reactor bed can be located in a third reactor.

In some aspects, the first input feed can include one or more first oxygenates, one or more first olefins, and methanol.

In some aspects, the first input feed can include one or more first oxygenates and methanol.

In some aspects, the first input feed can include one or more first oxygenates and one or more first olefins.

In some aspects, the one or more second oxygenates do not include methanol.

In some aspects, the one or more third oxygenates do not include methanol.

In some aspects, the one or more first olefins can include one or more recycled olefins, in which the one or more recycled olefins can include ethylene, propylene, butenes, pentenes, or any combination thereof.

### DETAILED DESCRIPTION

Certain exemplary aspects will now be described to provide an overall understanding of the principles of the structure, function, manufacture, and use of the systems and processes disclosed herein. One or more examples of these aspects are illustrated in the accompanying drawings. Those skilled in the art will understand that the systems and processes specifically described herein and illustrated in the accompanying drawings are non-limiting exemplary embodiments and that the scope of the present invention is not defined solely by the claims. The features illustrated or described in connection with one exemplary aspect may be combined with the features of other aspects. Such modifications and variations are intended to be included within the scope of the present invention.

Dehydration of alcohols to olefins (e.g. ethanol to ethylene) is particularly challenging to accomplish in cost effective manner. This is a significant issue, by way of example, as the production of ethylene from ethanol is the first step in the production of much of the available volume renewable chemicals (e.g., biobased polyethylene or biobased ethylene glycol) as well as the first step in multiple commercial processes to produce renewable hydrocarbon fuels from ethanol, especially sustainable aviation fuel (SAF). As dehydration reactions are highly endothermic, multiple reaction stages, very high recycle levels, or low feed concentrations are generally required to achieve high rates of conversion. All of the conventional approaches lead to both very high equipment costs and energy usage.

In the present systems and processes described herein, one or more oxygenates (e.g., C1+ alcohol(s), dimethylether, or mixtures thereof), and, optionally, an olefin co-feed, are converted to a mixture of olefins. The conversion process involves a combination of endothermic alcohol or ether dehydration to olefins followed by a net exothermic oligomerization and cracking of the resulting lower carbon number olefins to higher carbon number olefins. To maximize the potential for heat integration between these reactions and minimize the cost of equipment, as well as overall process heat demand, the present systems and processes are designed to control the relative rates of these two reactions to balance the heat released or adsorbed from the overall process by way of staged addition of the one or more oxygenates while the overall process is being carried out.

In the present disclosure, an adiabatic multistage reactor is designed to balance endothermic oxygenate dehydration and exothermic olefin oligomerization by splitting the oxygenate feed across multiple stages in an adiabatic reactor in such a way to use the endothermic oxygenate dehydration reaction to off-set the heat generated by the exothermic olefin oligomerization process and maintain the internal reactor temperatures within the desired limits across the reactor beds. As such, the conversion process across the present adiabatic multistage reactors therefore occurs at a more consistent temperature profile compared to conventional conversion processes that involve only a single dose of oxygenate feed. Further, this heat off-set prevents the need for either inter-stage removal of the material from the reactor during use, significant recycle of unconverted or partially converted oxygenates, or excessive dilution of the oxygenate feed, which would otherwise be needed if the oxygenate feed was provided as a single dose. It should be noted that reactor contents can be cooled or heated without heat removal or addition by supplying a secondary feed at a lower or higher temperature than the reactor contents at that stage.

In general, the present systems disclosed herein for converting one or more oxygenates to one or more olefins include an adiabatic multistage reactor having multiple inputs (e.g., at least first and second input feeds), at least a first reaction stage and a second reaction stage, where the first reaction stage is upstream of the second reaction stage. The first reaction stage includes a first reactor bed and the second reaction stage includes a second reactor bed. In use, a first input feed can be fed into a first end (e.g., an inlet) of the adiabatic multistage reactor and subsequently contact the first reactor bed to produce a first reaction mixture. A second input feed can be introduced into the adiabatic multistage reactor downstream of the first reaction stage, which then mixes with the first reaction mixture to produce a first effluent having a different composition relative to the first reaction mixture. The first effluent can then subsequently contact the second reactor bed to produce a second reaction mixture. This second reaction mixture can then be pass through the output of reactor, or in other instances to a subsequent reaction stage (e.g., third reaction stage) of the reactor.

In some aspects, during each reaction stage and between reaction stages, external heat is not added to the adiabatic multistage reactor. The phrase “external heat” is heat that is provided to the adiabatic multistage reactor that is not otherwise generated by a chemical reaction within the adiabatic multistage reactor or provided by any input feed (e.g., the first, second, or third input feeds). Alternatively, or in addition, during each reaction stage and between reaction stages, heat is not removed from the adiabatic multistage reactor.

In some aspects, the first effluent is not removed from the adiabatic multistage reactor. The compositional makeup of the first effluent can include water, oxygenates, and co-products. Alternatively, or in addition, in some aspects, the second effluent is not removed from the adiabatic multistage reactor. The compositional makeup of the second effluent can include water, oxygenates, olefins, and co-products (e.g., paraffins and aromatics).

In some aspects, heat is not removed from the first reaction mixture prior to being mixed with the second input feed. Alternatively, or in addition, in some aspects, heat is not removed from the second reaction mixture prior to being removed from the adiabatic multistage reactor or subsequently mixed with a third input feed.

The first input feed includes one or more first oxygenates. For the purposes of this disclosure, an “oxygenate” is a hydrocarbon that contains oxygen as part of its chemical structure. Non-limiting examples of first oxygenates include methanol, ethanol, butanols, pentanols, one or more esters, and/or one or more ethers. In some aspects, the one or more first oxygenates does not include methanol. In some aspects, the one or more first oxygenates can include the same oxygenate, and in other aspects, the one or more first oxygenates can include a mixture of different oxygenates. By way of example, in some aspects, the one or more first oxygenates can include a predominant first oxygenate, for example, ethanol. In such aspects, the one or more first oxygenates can also include one or more other oxygenates, e.g., methanol, propanol, one or more esters and/or one or more ethers. As used herein, a “predominant first oxygenate” can be present at a greater weight percent than any other individual oxygenate in the one or more first oxygenates, for example, present in an amount that is at least 25 weight percent, at least 50 weight percent, or at least 75 weight percent of the one or more first oxygenates. In some aspects, the predominant first oxygenate can be present in an amount of 25 weight percent to 99 weight percent of the one or more first oxygenates, in an amount of 25 weight percent to 90 weight percent of the one or more first oxygenates, in an amount of 50 weight percent to 99 weight percent of the one or more first oxygenates, or in an amount of 75 weight percent to 99 weight percent of the one or more first oxygenates. It is further contemplated that the predominant first oxygenate can be present between any of these recited ranges.

In some aspects, the one or more first oxygenates can include ethanol and methanol. Alternatively, or in addition, the one or more first oxygenates can include dimethylether. Further, the molar ratio of methanol to ethanol in the first input feed can be from about 0.5 to 6 or the molar ratio can be from about 1 to 5 or the molar ratio can be from about 2 to 5 or the molar ratio can be from about 3 to 6 or the molar ratio can be from about 3 to 5.

The one or more first oxygenates can be introduced into the adiabatic multistage reactor at a variety of temperatures. For example, in some aspects, the temperature of the one or more first oxygenates can be from about 300° C. to 550° C. or from about 400° C. to 500° C. In one aspect, the temperature of the one or more first oxygenates can be from about 300° C. to 480° C. In another aspect, the temperature of the one or more first oxygenates can be from about 480° C. to 550° C. or from about 450° C. to 500° C. It is also contemplated that the temperature of the one or more first oxygenates does not fall outside any of these recited ranges. It is further contemplated that the temperature of the one or more first oxygenates can be between any of these recited ranges.

The first input feed can also include other materials, such as, for example, one or more first olefins. Non-limiting examples of first olefins include ethylene, propylene, butenes, and the like. In some aspects, the one or more first olefins can include the same olefin, and in other aspects, the one or more first olefins can include a mixture of different olefins. By way of example, in some aspects, the one or more first olefins can include a predominant first olefin, for example, ethylene. As used herein, a “predominant first olefin” can be present at a greater weight percent than any other individual olefin in the one or more first olefins, for example, present in an amount that is at least 25 weight percent, at least 50 weight percent, or at least 75 weight percent of the one or more first olefins. In some aspects, the predominant first olefin can be present in an amount of 25 weight percent to 99 weight percent of the one or more first olefins, in an amount of 25 weight percent to 90 weight percent of the one or more first olefins, in an amount of 50 weight percent to 99 weight percent of the one or more first olefins, or in an amount of 75 weight percent to 99 weight percent of the one or more first olefins. It is further contemplated that the predominant first olefin can be present between any of these recited ranges. In some aspects, the first input feed can include ethanol and ethylene. For example, the molar ratio of ethylene:ethanol can be from about 0.25 to 10 in the first input feed or the molar ratio of ethylene:ethanol can be from about 0.25 to 5 in the first input feed.

In some aspects, at least one of the one or more first olefins are provided by way of an olefin recycle within the adiabatic multistage system. Such olefins are referred to herein as a “recycled oefin”). Non-limiting examples of suitable recycled olefins include ethylene, propylene, butenes, pentenes, or any combination thereof. By way of example, in some aspects at least one of the one or more first olefin can include an ethylene, alone or in combination with other olefins, e.g., one or more C3+ olefins, by way of recycling the ethylene produced within the adiabatic multistage reactor and combining with one or more first oxygenates (e.g., ethanol) to form the first input feed. In some aspects, at least one of the one or more first olefins can include recycled a mixture of C2-C4 olefins, whereas in other aspects, at least one of the one or more first olefins can include C2-C5 olefins.

The first input feed can be introduced into the adiabatic multistage reactor at a variety of temperatures. For example, in some aspects, the temperature of the first input feed can be from about 300° C. to 550° C. or from about 400° C. to 500° C. It is also contemplated that the temperature of the first input feed does not fall outside any of these recited ranges. It is further contemplated that the temperature of the first input feed can be between any of these recited ranges.

In use, the compositional makeup of the first input feed is designed to maintain a first temperature of the first reactor bed within a first temperature range. In some aspects, the first temperature range can be from about 300° C. to 550° C. In some aspects, the first temperature range can be from about 300° C. to 500° C., from about 350° C. to 500° C., from about 300° C. to 400° C., about 350° C. to 450° C., from about 400° C. to 460° C., from about 400° C. to 480° C., or from about 370° C. to 480° C. It is also contemplated that the first temperature does not fall outside any of these recited ranges. It is further contemplated that the first temperature can be between any of these recited ranges.

In addition to the compositional makeup of the first input feed, the temperature of the first reactor bed can also be dependent at least upon the compositional makeup of the first reactor bed. The first reactor bed can include one or more first catalysts. Thus, in some aspects, the first reactor bed can include a single catalyst, whereas in other aspects, the first reactor bed includes a mixture of two or more catalysts. It should be noted that the first reactor bed should be designed so as to avoid over converting the one or more first oxygenates and the one or more olefins (e.g., lower carbon olefins) present within the first input feed, which would overcool or overheat the first reaction stage, respectively. Thus, the compositional makeup of the first reactor bed can be designed based on the desired rate and composition of the one or more first olefins relative to the total flow of the one or more first oxygenates.

In some aspects, the one or more first catalysts include a doped or undoped zeolite catalyst. Non-limiting examples of suitable zeolite catalysts include pentasil types, such as ZSM-5, (MFI or BEA frameworks), CHA, FER, FAU, MWW, MOR, EUO, MFS, ZSM-48, MTT or TON having Si/Al higher than 10, or a dealuminated crystalline silicate of the group ZSM5 (MFI or BEA frameworks), CHA, FER, FAU, MWW, MOR, EUO, MFS, ZSM-48, MTT or TON having Si/Al higher than 10, or a phosphorus and/or boron modified crystalline silicate of the group ZSM-5 (MFI or BEA frameworks), CHA, FER, FAU, MWW, MOR, EUO, MFS, ZSM-48, MTT or TON having Si/Al higher than 10, or molecular sieves of the type silico-aluminophosphate of the group AEL. Non-limiting examples of suitable dopants of the zeolite catalyst include phosphorus and/or boron. In some aspects, the zeolite catalyst can be a boron and phosphorous doped zeolite. Additional additives for mixing with doped zeolites include SiO₂ supports doped with metal dopants can include sodium (Na), potassium (K), lithium (Li), beryllium (Be), magnesium (Mg), calcium (Ca), strontium (Sr), barium, radium, iron (Fe), cobalt (Co), nickel (Ni), lanthanum (La), chromium (Cr), zirconium (Zr), ruthenium (Ru), molybdenum (Mo), iridium (Ir), tungsten (W), copper (Cu), manganese (Mn), vanadium (V,) zinc (Zn), titanium (Ti), rhodium (Rh), rhenium (Re), gallium (Ga), palladium (Pd), silver (Ag), indium (In), or any combinations thereof.

In further aspects, the one or more first catalysts can also include an alcohol or ether dehydration specific catalyst, e.g., solid acids, a doped or undoped alumina, such as zirconated alumina, gamma-alumina, high purity gamma-alumina, or doped gamma-alumina, or a doped or undoped zeolites with limited olefin oligomerization activity (e.g., where such a zeolite would dehydrate an alcohol to its corresponding olefin with at least 80 mol % selectively under the applied conditions), for example, H-MFI type zeolites with high Si/Al₂ ratios (e.g. >190) or that have been dealuminated, and under certain conditions, Si/Al₂ ratios H-FER, H-BEA, or H—Y type zeolites can also be considered monofunctional dehydration catalysts.

Exemplary catalyst combinations, physically mixed within the first reactor bed can include one part (e.g., a first catalyst of the one or more first catalysts) doped zeolites such as crystalline silicates of the group ZSM-5 (MFI or BEA frameworks), CHA, FER, FAU, MWW, MOR, EUO, MFS, ZSM-48, MTT or TON having Si/Al higher than 10, or a dealuminated crystalline silicate of the group ZSM5 (MFI or BEA frameworks), CHA, FER, FAU, MWW, MOR, EUO, MFS, ZSM-48, MTT or TON having Si/Al higher than 10, or a phosphorus and/or boron modified crystalline silicate of the group ZSM-5 (MFI or BEA frameworks), CHA, FER, FAU, MWW, MOR, EUO, MFS, ZSM-48, MTT or TON having Si/Al higher than 10, or molecular sieves of the type silico-aluminophosphate of the group AEL. Additional additives for mixing with doped zeolites consist of SiO₂ supports doped with metal dopants including iron (Fe), strontium (Sr), cobalt (Co), nickel (Ni), lanthanum (La), chromium (Cr), zirconium (Zr), ruthenium (Ru), molybdenum (Mo), iridium (Ir), magnesium (Mg), tungsten (W), copper (Cu), manganese (Mn), vanadium (V,) zinc (Zn), titanium (Ti), rhodium (Rh), rhenium (Re), gallium (Ga), palladium (Pd), silver (Ag), indium (In), or any combination thereof. A second part of the catalyst mixture (e.g., a second catalyst of the one or more first catalysts) can include an alcohol or ether dehydration specific catalyst as discussed above.

The first reactor bed can have a variety of structural configurations. For example, in some aspects, the first reactor bed is a fixed reactor bed. In some aspects, the fixed bed reactor is an axial flow fixed bed reactor. In some aspects, the fixed bed reactor is a radial flow fixed bed reactor. In other aspects, the first reactor bed is a fluidized bed. In yet other aspects, the first reactor bed is a moving bed.

The second input feed includes one or more second oxygenates, so-called as they are the second oxygenates introduced into the reactor at the second stage or reactor bed. Non-limiting examples of second oxygenates include methanol, ethanol, butanols, pentanols, one or more esters, and/or one or more ethers. In some aspects, the one or more second oxygenates does not include methanol. In some aspects, the one or more second oxygenates can include the same oxygenate, and in other aspects, the one or more second oxygenates can include a mixture of different oxygenates. By way of example, in some aspects, the one or more second oxygenates can include a second predominant oxygenate, for example, ethanol. As used herein, a “predominant second oxygenate” can be present at a greater weight percent than any other individual oxygenate in the one or more second oxygenates, for example, present in an amount that is at least 25 weight percent, at least 50 weight percent, or at least 75 weight percent of the one or more second oxygenates. In some aspects, the predominant second oxygenate can be present in an amount of 25 weight percent to 99 weight percent of the one or more second oxygenates, in an amount of 25 weight percent to 90 weight percent of the one or more second oxygenates, in an amount of 50 weight percent to 99 weight percent of the one or more second oxygenates, or in an amount of 75 weight percent to 99 weight percent of the one or more second oxygenates. It is further contemplated that the predominant second oxygenate can be present between any of these recited ranges.

In general, the one or more second oxygenates are the same as the one or more first oxygenates such that overall conversion process within the adiabatic multistage reactor includes two or more injections of oxygenates, and more specifically, a separate injection of oxygenate at different reaction stages. This allows the system to control the temperatures of the reactor beds, thereby maximizing heat integration across the adiabatic multistage reactor. In some aspects, the predominant second oxygenate of the one or more second oxygenates can include ethanol. In such aspects, the one or more second oxygenates can also include one or more other oxygenates, e.g., methanol, propanols, butanols, pentanols, one or more esters, and/or one or more ethers.

The one or more second oxygenates can be introduced into the adiabatic multistage reactor at a variety of temperatures. For example, in some aspects, the temperature of the one or more second oxygenates can be from about 300° C. to 550° C. or from about 400° C. to 500° C. In one aspect, the temperature of the one or more second oxygenates can be from about 300° C. to 480° C. In another aspect, the temperature of the one or more second oxygenates can be from about 480° C. to 550° C. or from about 450° C. to 500° C. It is also contemplated that the temperature of the one or more second oxygenates does not fall outside any of these recited ranges. It is further contemplated that the temperature of the one or more second oxygenates can be between any of these recited ranges.

The second input feed can be introduced into the adiabatic multistage reactor at a variety of temperatures. For example, in some aspects, the temperature of the second input feed can be from about 200° C. to 550° C., from about 200° C. to 500° C., from about 300° C. to 500° C., from about 360° C. to 400° C., from about 400° C. to 500° C., or from about 450° C. to 550° C. It is also contemplated that the temperature of the one or more second input feed does not fall outside any of these recited ranges. It is further contemplated that the temperature of the second input feed can be between any of these recited ranges. In some aspects, the second input feed is introduced into the adiabatic multistage reactor at a temperature that can be greater than a temperature of the first reaction mixture

In use, the compositional makeup of the second input feed is designed to maintain a second temperature of the second reactor bed within a second temperature range. In some aspects, the second temperature range can be from about 300° C. to 550° C. In some aspects, the second temperature range can be from about 300° C. to 500° C., from about 350° C. to 500° C., from about 300° C. to 400° C., from about 350° C. to 450° C., from about 400° C. to 480° C., or from about 380° C. to 450° C. It is also contemplated that the second temperature does not fall outside any of these recited ranges. It is further contemplated that the second temperature can be between any of these recited ranges.

In addition to the compositional makeup of the second input feed, the temperature of the second reactor bed can also be dependent at least upon the compositional makeup of a bifunctional catalyst or specific admixture of catalysts in the second reactor bed. The second reactor bed can include one or more second catalysts. Thus, in some aspects, the second reactor bed can include a single catalyst, whereas in other aspects, the second reactor bed includes a mixture of two or more mono- or bifunctional types of catalysts. It should be noted that the second reactor bed should be designed so as to avoid over converting the one or more second oxygenates and the one or more second olefins (e.g., lower carbon olefins) present within the first reaction mixture, which would overcool or overheat the second reaction stage, respectively. Thus, the compositional makeup of the second reactor bed can be designed based on the desired rate and composition of the one or more second olefins relative to the total flow of the one or more second oxygenates.

In some aspects, the one or more second catalysts include a doped or undoped zeolite catalyst. Non-limiting examples of suitable zeolite catalysts include pentasil types, such as ZSM-5, (MFI or BEA frameworks), CHA, FER, FAU, MWW, MOR, EUO, MFS, ZSM-48, MTT or TON having Si/Al higher than 10, or a dealuminated crystalline silicate of the group ZSM5 (MFI or BEA frameworks), CHA, FER, FAU, MWW, MOR, EUO, MFS, ZSM-48, MTT or TON having Si/Al higher than 10, or a phosphorus and/or boron modified crystalline silicate of the group ZSM-5 (MFI or BEA frameworks), CHA, FER, FAU, MWW, MOR, EUO, MFS, ZSM-48, MTT or TON having Si/Al higher than 10, or molecular sieves of the type silico-aluminophosphate of the group AEL. Non-limiting examples of suitable dopants of the zeolite catalyst include phosphorus and/or boron. Additional additives for mixing with doped zeolites include SiO₂ supports doped with metal dopants can include sodium (Na), potassium (K), lithium (Li), beryllium (Be), magnesium (Mg), calcium (Ca), strontium (Sr), barium, radium, iron (Fe), cobalt (Co), nickel (Ni), lanthanum (La), chromium (Cr), zirconium (Zr), ruthenium (Ru), molybdenum (Mo), iridium (Ir), tungsten (W), copper (Cu), manganese (Mn), vanadium (V,) zinc (Zn), titanium (Ti), rhodium (Rh), rhenium (Re), gallium (Ga), palladium (Pd), silver (Ag), and/or indium (In).

In further aspects, the one or more second catalysts can also include an alcohol or ether dehydration specific catalyst, e.g., solid acids, a doped or undoped alumina, such as zirconated alumina, gamma-alumina, high purity gamma-alumina, or doped gamma-alumina, or a doped or undoped zeolites with limited olefin oligomerization activity (e.g., where such a zeolite would dehydrate an alcohol to its corresponding olefin with at least 80 mol % selectively under the applied conditions), for example, H-MFI type zeolites with high Si/Al₂ ratios (e.g. >190) or that have been dealuminated, and under certain conditions, Si/Al₂ ratios H-FER, H-BEA, or H—Y type zeolites can also be considered monofunctional dehydration catalysts.

Exemplary catalyst combinations, physically mixed within the second reactor bed can include one part (e.g., a first catalyst of the one or more second catalysts) doped zeolites such as crystalline silicates of the group ZSM-5 (MFI or BEA frameworks), CHA, FER, FAU, MWW, MOR, EUO, MFS, ZSM-48, MTT or TON having Si/Al higher than 10, or a dealuminated crystalline silicate of the group ZSM5 (MFI or BEA frameworks), CHA, FER, FAU, MWW, MOR, EUO, MFS, ZSM-48, MTT or TON having Si/Al higher than 10, or a phosphorus and/or boron modified crystalline silicate of the group ZSM-5 (MFI or BEA frameworks), CHA, FER, FAU, MWW, MOR, EUO, MFS, ZSM-48, MTT or TON having Si/Al higher than 10, or molecular sieves of the type silico-aluminophosphate of the group AEL. Additional additives for mixing with doped zeolites consist of SiO₂ supports doped with metal dopants including iron (Fe), strontium (Sr), cobalt (Co), nickel (Ni), lanthanum (La), chromium (Cr), zirconium (Zr), ruthenium (Ru), molybdenum (Mo), iridium (Ir), magnesium (Mg), tungsten (W), copper (Cu), manganese (Mn), vanadium (V,) zinc (Zn), titanium (Ti), rhodium (Rh), rhenium (Re), gallium (Ga), palladium (Pd), silver (Ag), and/or indium (In). A second part of the catalyst mixture (e.g., a second catalyst of the one or more first catalysts) can include an alcohol or ether dehydration specific catalyst as discussed above.

The second reactor bed can have a variety of structural configurations. For example, in some aspects, the second reactor bed is a fixed reactor bed. In some aspects, the fixed bed reactor is an axial flow fixed bed reactor. In some aspects, the fixed bed reactor is a radial flow fixed bed reactor. In other aspects, the second reactor bed is a fluidized bed. In yet other aspects, the second reactor bed is a moving bed.

In some aspects, the adiabatic multistage reactor can include one or more additional reaction stages, each having a respective reactor bed. For example, the adiabatic multistage reactor can include a third reaction stage having a third reactor bed. The third reactor bed can have a variety of configurations. For example, in some aspects, the third reactor bed is a fixed reactor bed. In some aspects, the fixed bed reactor is an axial flow fixed bed reactor. In some aspects, the fixed bed reactor is a radial flow fixed bed reactor. In other aspects, the third reactor bed is a fluidized bed. In yet other aspects, the third reactor bed is a moving bed.

In use, a third input feed is introduced into the adiabatic multistage reactor downstream of the second reaction stage such that, upon exiting the second reaction stage, the second reaction mixture can be mixed with the third input feed to produce a second effluent having a different composition relative to the second reaction mixture. The second effluent then contacts the third reactor bed to thereby maintain a third temperature of the third reactor bed within a third temperature range from about 300° C. to 550° C. and to produce a third reaction mixture. In some aspects, heat is not removed from the third reaction mixture prior to being removed from the adiabatic multistage reactor or subsequently mixed with a fourth input feed.

The third input feed includes one or more third oxygenates, so-called as they are the third oxygenates introduced into the reactor at the third stage or reactor bed. Non-limiting examples of third oxygenates include methanol, ethanol, propanols, butanols, pentanols, one or more esters, and/or one or more ethers. In some aspects, the one or more third oxygenates does not include methanol. In some aspects, the one or more third oxygenates can include the same oxygenate, and in other aspects, the one or more third oxygenates can include a mixture of different oxygenates. By way of example, in some aspects, the one or more third oxygenates can include a predominant third oxygenate, for example, ethanol. As used herein, a “predominant third oxygenate” can be present at a greater weight percent than any other individual oxygenate in the one or more third oxygenates, for example, present in an amount that is at least 25 weight percent, at least 50 weight percent, or at least 75 weight percent of the one or more third oxygenates. In some aspects, the predominant third oxygenate can be present in an amount of 25 weight percent to 99 weight percent of the one or more third oxygenates, in an amount of 25 weight percent to 90 weight percent of the one or more third oxygenates, in an amount of 50 weight percent to 99 weight percent of the one or more third oxygenates, or in an amount of 75 weight percent to 99 weight percent of the one or more third oxygenates. It is further contemplated that the predominant third oxygenate can be present between any of these recited ranges.

In general, the one or more third oxygenates are the same as the one or more first oxygenates and the one or more second oxygenates such that overall conversion process within the adiabatic multistage reactor includes two or more injections of oxygenates, and more specifically, a separate injection of oxygenate at different reaction stages. This further allows the system to control the temperatures of the reactor beds, thereby maximizing heat integration across the adiabatic multistage reactor. In some aspects, the predominant third oxygenate of the one or more third oxygenates can include ethanol. In such aspects, the one or more third oxygenates can also include one or more other oxygenates, e.g., methanol, propanol, one or more esters, and or one or more ethers.

The one or more third oxygenates can be introduced into the adiabatic multistage reactor at a variety of temperatures. For example, in some aspects, the temperature of the one or more third oxygenates can be from about 300° C. to 550° C. or from about 400° C. to 500° C. In one aspect, the temperature of the one or more third oxygenates can be from about 300° C. to 480° C. In another aspect, the temperature of the one or more third oxygenates can be from about 480° C. to 550° C. or from about 450° C. to 500° C. It is also contemplated that the temperature of the one or more third oxygenates does not fall outside any of these recited ranges. It is further contemplated that the temperature of the one or more third oxygenates can be between any of these recited ranges.

The third input feed can be introduced into the adiabatic multistage reactor at a variety of temperatures. For example, in some aspects, the temperature of the third input feed can be from about 200° C. to 550° C., from about 200° C. to 500° C., or from about 300° C. to 500° C. In some aspects, the third input feed is introduced into the adiabatic multistage reactor at a temperature that can be greater than a temperature of the second reaction mixture.

In use, the compositional makeup of the third input feed is designed to maintain a third temperature of the third reactor bed within a third temperature range. In some aspects, the third temperature range can be from about 300° C. to 550° C. In some aspects, the third temperature range can be from about 300° C. to 500° C., from about 350° C. to 500° C., from about 300° C. to 400° C., from about 350° C. to 450° C., from about 400° C. to 460° C., from about 400° C. to 480° C., or from about 370° C. to 480° C. It is also contemplated that the third temperature does not fall outside any of these recited ranges. It is further contemplated that the second temperature can be between any of these recited ranges.

In addition to the compositional makeup of the third input feed, the temperature of the third reactor bed can also be dependent at least upon the compositional makeup of the third reactor bed. The third reactor bed can include one or more third catalysts (e.g., bifunctional-type catalyst(s)). Thus, in some aspects, the third reactor bed can include a single catalyst, whereas in other aspects, the third reactor bed includes a mixture of two or more catalysts. It should be noted that the third reactor bed should be designed so as to avoid over converting the one or more third oxygenates and the one or more third olefins (e.g., lower carbon olefins) present within the second reaction mixture, which would overcool or overheat the third reaction stage, respectively. Thus, the compositional makeup of the third reactor bed can be designed based on the desired rate and composition of the one or more third olefins relative to the total flow of the one or more third oxygenates.

In some aspects, the one or more third catalysts include a doped or undoped zeolite catalyst. Non-limiting examples of suitable zeolite catalysts include pentasil types, such as ZSM-5, (MFI or BEA frameworks), CHA, FER, FAU, MWW, MOR, EUO, MFS, ZSM-48, MTT or TON having Si/Al higher than 10, or a dealuminated crystalline silicate of the group ZSM5 (MFI or BEA frameworks), CHA, FER, FAU, MWW, MOR, EUO, MFS, ZSM-48, MTT or TON having Si/Al higher than 10, or a phosphorus and/or boron modified crystalline silicate of the group ZSM-5 (MFI or BEA frameworks), CHA, FER, FAU, MWW, MOR, EUO, MFS, ZSM-48, MTT or TON having Si/Al higher than 10, or molecular sieves of the type silico-aluminophosphate of the group AEL. Non-limiting examples of suitable dopants of the zeolite catalyst include phosphorus and/or boron. In some aspects, the zeolite catalyst can be a boron and phosphorous doped zeolite. Additional additives for mixing with doped zeolites include SiO₂ supports doped with metal dopants can include sodium (Na), potassium (K), lithium (Li), beryllium (Be), magnesium (Mg), calcium (Ca), strontium (Sr), barium, radium, iron (Fe), cobalt (Co), nickel (Ni), lanthanum (La), chromium (Cr), zirconium (Zr), ruthenium (Ru), molybdenum (Mo), iridium (Ir), tungsten (W), copper (Cu), manganese (Mn), vanadium (V,) zinc (Zn), titanium (Ti), rhodium (Rh), rhenium (Re), gallium (Ga), palladium (Pd), silver (Ag), and/or indium (In).

In further aspects, the one or more third catalysts can also include an alcohol or ether dehydration specific catalyst, e.g., solid acids, a doped or undoped alumina, such as zirconated alumina, gamma-alumina, high purity gamma-alumina, or doped gamma-alumina, or a doped or undoped zeolites with limited olefin oligomerization activity, (e.g., where such a zeolite would dehydrate an alcohol to its corresponding olefin with at least 80 mol % selectively under the applied conditions), for example, H-MFI type zeolites with high Si/Al₂ ratios (e.g. >190) or that have been dealuminated, and under certain conditions, Si/Al₂ ratios H-FER, H-BEA, or H—Y type zeolites can also be considered monofunctional dehydration catalysts.

Exemplary catalyst combinations, physically mixed within the third reactor bed can include one part (e.g., a first catalyst of the one or more third catalysts) doped zeolites such as crystalline silicates of the group ZSM-5 (MFI or BEA frameworks), CHA, FER, FAU, MWW, MOR, EUO, MFS, ZSM-48, MTT or TON having Si/Al higher than 10, or a dealuminated crystalline silicate of the group ZSM5 (MFI or BEA frameworks), CHA, FER, FAU, MWW, MOR, EUO, MFS, ZSM-48, MTT or TON having Si/Al higher than 10, or a phosphorus and/or boron modified crystalline silicate of the group ZSM-5 (MFI or BEA frameworks), CHA, FER, FAU, MWW, MOR, EUO, MFS, ZSM-48, MTT or TON having Si/Al higher than 10, or molecular sieves of the type silico-aluminophosphate of the group AEL. Additional additives for mixing with doped zeolites consist of SiO₂ supports doped with metal dopants including iron (Fe), strontium (Sr), cobalt (Co), nickel (Ni), lanthanum (La), chromium (Cr), zirconium (Zr), ruthenium (Ru), molybdenum (Mo), iridium (Ir), magnesium (Mg), tungsten (W), copper (Cu), manganese (Mn), vanadium (V,) zinc (Zn), titanium (Ti), rhodium (Rh), rhenium (Re), gallium (Ga), palladium (Pd), silver (Ag), and/or indium (In). A second part of the catalyst mixture (e.g., a second catalyst of the one or more first catalysts) can include an alcohol or ether dehydration specific catalyst as discussed above.

As noted above, the adiabatic multistage reactor can contain any number of reaction stages equal to or greater than two in order to achieve a desired output composition. As such, in further aspects, the multistage reactor includes one or more additional reaction stages downstream of the third reaction stage. The one or more additional reaction stages each have a respective reactor bed. In use, a subsequent input feed is introduced into the multistage reactor downstream of prior reaction stages (e.g., first, second, and third reaction stages) such that, upon exiting the prior reaction stage (e.g., the third reaction stage), a prior reaction mixture (e.g., a third reaction mixture) is mixed with the subsequent input feed to produce an additional effluent having a different composition than the prior reaction mixture. The subsequent input feed can include one or more additional oxygenates. The prior effluent can then contact the respective reactor bed of one of the one of more additional reaction stages to thereby maintain a temperature of the respective reactor bed within a temperature range (e.g., from about 300° C. to 550° C.) and to produce an additional reaction mixture downstream of the prior reaction stages.

While the foregoing discussion of the adiabatic multistage reactor includes all beds in single adiabatic multistage reactor, it is also contemplated herein that each reactor bed can be located with a separate adiabatic reactor. For example, in some aspects, the first, second, and third reactor beds discussed above are each located in a respective adiabatic reactor. Such an approach would allow the use of moving beds, as well as provide the ability to specifically access and service or replace each bed individually. It is further contemplated herein where two or more adiabatic reactors are implemented where at least of the two or more adiabatic reactors includes two or more catalysts bed. For example, in some aspects, a first adiabatic reactor includes a first reactor bed and a second reactor bed and a second adiabatic reactor includes a third reactor bed and optionally, a fourth reactor bed.

In instances where there are two or more adiabatic reactors, these reactors can be collectively referred to herein as a reactor assembly (e.g., two or more adiabatic multistage reactors can be collectively referred to herein as an adiabatic multistage reactor assembly). In some aspects, a process for converting one or more oxygenates to one or more olefins, can include introducing a first input feed into a first end of adiabatic multistage reactor assembly, the multistage reactor assembly having at least a first reaction stage and a second reaction stage, wherein the first reaction stage is upstream of the second reaction stage, and the first reaction stage having a first reactor bed, and the second reaction stage having a second reactor bed. The first input feed can include one or more first oxygenates and at least one of one or more first olefins or methanol. The process can also include contacting the first input feed with the first reactor bed to thereby maintain a first temperature of the first reactor bed within a first temperature range from about 300° C. to 550° C. and to produce a first reaction mixture, and introducing a second input feed into the multistage reactor assembly downstream of the first reaction stage such that, upon exiting the first reaction stage, the first reaction mixture is mixed with the second input feed to produce a first effluent having a different composition relative to the first reaction mixture. The second input feed can include one or more second oxygenates. The process can also further include contacting the first effluent with the second reactor bed to thereby maintain a second temperature of the second reactor bed within a second temperature range from about 300° C. to 550° C. and to produce a second reaction mixture.

In some aspects, the multistage reactor assembly can include a third reaction stage, in which the third reaction stage has a third reactor bed. In such aspects, the process can further include introducing a third input feed into the multistage reactor system downstream of the second reaction stage such that, upon exiting the second reaction stage, the second reaction mixture is mixed with the third input feed to produce a second effluent having a different composition relative to the second reaction mixture. The third input feed can include one or more third oxygenates. The process can also include contacting the second effluent with the third reactor bed to thereby maintain a third temperature of the third reactor bed within a third temperature range from about 300° C. to 550° C. and to produce a third reaction mixture.

In some aspects, the multistage reactor assembly can include two or more reactors, in which the first and second stages can be carried in the first reactor and the third stage can be carried out in the second reactor.

In some aspects, the multistage reactor assembly can include two or more reactors, in which the first reactor bed can be located in a first reactor and the second reactor bed can be located in a second reactor. In such aspects, the third reactor bed can be located in a third reactor.

In aspects where the adiabatic multistage reactor includes only two reactor beds, the output of the adiabatic multistage reactor is the second reaction mixture. The compositional makeup of the second reaction mixture can include oxygenates, e.g., alcohol(s) and/or ether(s), olefins, e.g., C5+ olefins, water, and co-product(s), e.g., saturate(s). In some aspects, the alcohols(s) include ethanol, propanols, butanols, and the like.

In aspects where the adiabatic multistage reactor includes three reactor beds, the output of adiabatic multistage reactor is the third reaction mixture. The compositional makeup of the third reaction mixture can include oxygenates, e.g., alcohol(s) and/or ether(s), olefins, e.g., C5+ olefins, water, and co-product(s), e.g., saturate(s). In some aspects, the alcohols(s) include ethanol, propanols, butanols, and the like.

The present systems for conversion of oxygenates can include additional elements. For example, the present systems can include, in addition to the adiabatic multistage reactor, one or more of the following: a single stage reactor, one or more heat exchanger units, a condenser, a separation subsystem (e.g., a distillation system), one or more furnaces, or any combinations thereof. While not discussed in detail herein, it should be noted that further elements present within the system are also contemplated herein.

In some aspects, the system can include a single stage reactor comprising a reactor bed having one or more catalysts. In use, the output of the adiabatic multistage reactor (e.g., the second reaction mixture or the third reaction mixture), can be introduced into a single stage reactor and contacted with the one or more catalysts to produce an output stream comprising one or more product olefins. The one or more product olefins can include C2-C7 olefins, for example, ethylene, propylene, butenes, pentenes, and the like, or any combination thereof. In one aspect, the one or more product olefins can include at least ethylene.

In aspects where the adiabatic multistage reactor includes only two reactor beds, prior to introducing the second reaction mixture into the single stage reactor, the temperature of the second reaction mixture can be decreased. In aspects where the adiabatic multistage reactor includes only three reactor beds, prior to introducing the third reaction mixture into the single stage reactor, the temperature of the third reaction mixture can be decreased. In either instance, this reduction in temperature can be carried out, for example, by a heat exchanger unit.

In some aspects, the output stream can be introduced into a separation subsystem to produce a first stream and a second stream. In certain aspects, prior to introducing the output stream into the separation subsystem, the output stream can be condensed into a condensed output stream, and the condensed output stream can be introduced into the separation subsystem. The separation subsystem can include a variety of separation units, such as a distillation system, a liquid-liquid separation system, a liquid extraction system, a membrane separation system, and an adsorbent(s) system to carry out separation processes to produce the first and second streams.

The first stream can include ethylene, propylene, butane(s), or any combination thereof. In one aspect, the first stream can include a predominant olefin, for example, ethylene. As used herein, a “predominant olefin” can be present at a greater weight percent than any other individual olefin in the first stream, for example, present in an amount that is at least 50 weight percent, at least 75 weight percent, or at least 95 weight percent of the olefins within the first stream. In some aspects, the predominant olefin can be present in an amount of 25 weight percent to 99 weight percent of the olefins within the first stream, in an amount of 25 weight percent to 90 weight percent of the olefins within the first stream, in an amount of 35 weight percent to 90 weight percent of the olefins within the first stream, in an amount of 40 weight percent to 90 weight percent of the olefins within the first stream, in an amount of 45 weight percent to 90 weight percent of the olefins within the first stream, in an amount of 50 weight percent to 99 weight percent of the olefins within the first stream, in an amount of 55 weight percent to 99 weight percent of the olefins within the first stream, in an amount of 60 weight percent to 99 weight percent of the olefins within the first stream, or in an amount of 75 weight percent to 99 weight percent of the olefins within the first stream. In some aspects, the predominant olefin can be present in an amount of 35 weight percent to 65 weight percent of the olefins within the first stream. It is further contemplated that the predominant olefin can be present between any of these recited ranges.

The second stream can include C3+ olefins, such as at least one C3 to C7 olefin. In addition, in some aspects, the second stream can include co-products, such as aromatic(s) and saturate(s).

In aspects where an olefin recycle is incorporated within the system, the first stream can be combined with the one or more first oxygenates to produce the first input feed. In some aspects, prior to introducing the first input stream into the adiabatic multistage reactor, the one or more first oxygenates can be heated, for example, to a temperature from about 200° C. to 500° C., from about 200° C. to 500° C., from about 375° C. to 500° C., from about 360° C. to 550° C., or from about 400° C. to 500° C. Alternatively, or in addition, prior to introducing the second input stream into the adiabatic multistage reactor, the one or more second oxygenates can be heated, for example, to a temperature from about 200° C. to 500° C., from about 200° C. to 500° C., from about 375° C. to 500° C., from about 360° C. to 550° C., or from about 400° C. to 500° C. Alternatively, or in addition, prior to introducing the third input stream into the adiabatic multistage reactor, the one or more second oxygenates can be heated, for example, to a temperature from about 200° C. to 500° C., from about 200° C. to 500° C., from about 375° C. to 500° C., from about 360° C. to 550° C., or from about 400° C. to 500° C. In some aspects, the one or more first oxygenates, the one or more second oxygenates, and/or the one or more third oxygenates can be heated by way of a heat exchanger, e.g., a single furnace. In other aspects, the one or more first oxygenates, the one or more second oxygenates, and/or the one or more third oxygenates can be heated by way of two or more heat exchangers, e.g., respective furnaces.

In some aspects, the adiabatic multistage reactor can be at a weight hourly space velocity (WHSV) from about 0.25 to 15. As used herein, “weight hourly space velocity” is defined as the weight of hydrocarbon compounds flowing per hour per total weight of catalysts in the multistage reactor.

It should be appreciated that the net thermodynamics of the oxygenate(s) to olefin(s) process depends at least upon the identity of the oxygenate, the desired olefin product mixture, and the presence of any side reactions. For example, in some aspects, especially at higher recycle rates of lower olefins, the net process can be exothermic and heat needs to be removed from the system. This cooling can be accomplished in multiple ways. For example, a heat exchanger can be used to decrease the temperature of the output of the adiabatic multistage reactor prior to subjecting it to a final stage, or stages, of olefin oligomerization and cracking. This heat exchanger could be separate from the adiabatic multistage reactor or integrated within the reactor. The final stage(s) of reaction could be performed in a separate vessel, such as in a single reactor downstream of the adiabatic multistage reactor, or a side stream from the adiabatic multistage reactor could be cooled and then reintroduced into the adiabatic multistage reactor. Another approach would be to have lower temperature second and third oxygenate input feeds that cool the respect reactor beds.

In some aspects, especially at low to zero recycle rates of lower olefins or in instances where the first input feed includes lower amounts of methanol or lower olefins, the net process can be endothermic and heat would need to be added to the system. This heating can be accomplished in multiple ways. One approach would be to heat the one or more first oxygenates above the temperature of the first reactor bed. Some amount of recycle of the one or more olefins (e.g., lower olefin(s) recycle) may be needed to maintain process temperatures in the initial stage or stages of the adiabatic multistage reactor. Subsequent additions of one or more oxygenates (e.g., the one or more second oxygenates and/or the one or more third oxygenates) can be heated above the maximum desired temperature for the adiabatic multistage reactor to bring the total process temperature before each reaction stage high enough to continue to simultaneously dehydrate the one or more oxygenates and oligomerize the olefins present in the reaction mixtures (e.g., the first reaction mixture, the second reaction mixture, and/or the third reaction mixture). Net lower olefin recycle can be reduced, in some cases to near negligible values, by increasing the number of reaction stages, though selectivity toward olefins may be diminished as the olefins are exposed to the catalyst(s) for longer periods of time.

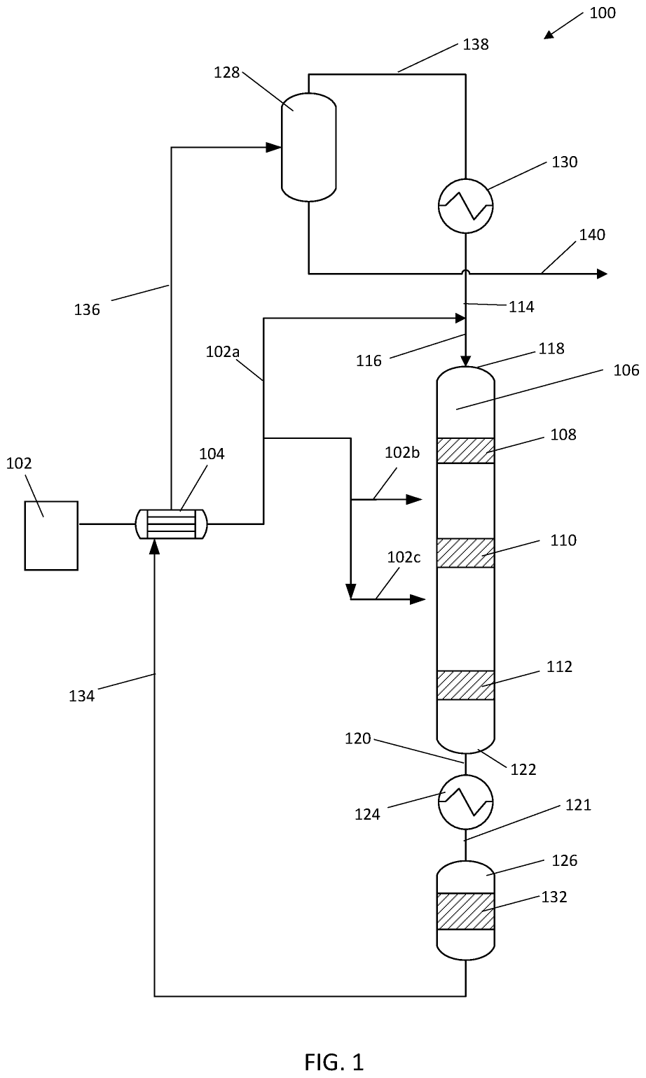

An exemplary schematic of a system 100 for conversion of oxygenates is illustrated in FIG. 1. For simplicity purposes only, this exemplary system 100 is discussed with respect to the one or more first, second, and third oxygenates being ethanol and the one or more olefins being ethylene. A person skilled in the art would appreciate that this exemplary system can be used for other oxygenates and olefins, and therefore is not limited to ethanol or ethylene.

In the illustrated system 100, the system 100 includes a source of ethanol 102, and an optional heat integration subsystem 104 and an adiabatic multistage reactor 106. While the adiabatic multistage reactor 106 can have 2 or more reaction stages, in this illustrated example, the adiabatic multistage reactor includes three reaction stages. The first reaction stage includes a first reactor bed 108, the second reaction stage includes a second reactor bed 110, and the third reaction stage includes a third reactor bed 112. Each reactor bed 108, 110, 112 includes one or more catalysts. As will be described in more detail below, in this illustrated system, the heated ethanol is provided to the adiabatic multistage reactor 106 in three portions. While the heat integration subsystem can have a variety of configurations, in this illustrated system, the heat integration subsystem includes a preheater that is configured to preheat the ethanol, e.g., to a temperature of about 450° C. and a condenser that is configured to condense an output stream (e.g., the output stream of the single stage reactor) to a condensed output stream.

In use, ethanol 102 is introduced into the preheater (not shown) and heated. A first portion of the heated ethanol 102a is then combined with an ethylene recycle stream 114 (e.g., a first stream) to generate a first input feed 116 that is then introduced into a first end 118 (e.g., a first inlet) of the adiabatic multistage reactor 106. The first input feed 116 then contacts the first reactor bed 108 to thereby maintain a first temperature of the first reactor bed 108 within a first temperature range and to produce a first reaction mixture (not shown). The first reactor bed 108 is designed such that the heat produced from exothermic olefin oligomerization expected is substantially, or completely, balanced with the heat absorbed by dehydration of the ethanol.

A second portion of the heated ethanol is introduced into the adiabatic multistage reactor 106 as a second input feed 102b. Upon exiting the first reaction stage, the first reaction mixture (not shown) is mixed with the second input feed 102b within the adiabatic multistage reactor 106 to produce a first effluent (not shown). The first effluent then contacts the second reactor bed 110 to thereby maintain a second temperature of the second reactor bed 110 within a second temperature range and to produce a second reaction mixture (not shown). The second reactor bed 110 is designed such that the heat produced from exothermic olefin oligomerization expected is substantially, or completely, balanced with the heat absorbed by dehydration of the ethanol to ethylene.

A third portion of the heated ethanol is introduced into the adiabatic multistage reactor 106 as a third input feed 102c. Upon exiting the second reaction stage, the second reaction mixture (not shown) is mixed with the second input feed 102c within the adiabatic multistage reactor 106 to produce a second effluent (not shown). The second effluent then contacts the third reactor bed 112 to thereby maintain a second temperature of the third reactor bed 112 within a third temperature range and to produce a third reaction mixture 120. The third reactor bed 112 is designed such that the heat produced from exothermic olefin oligomerization expected is substantially, or completely, balanced with the heat absorbed by dehydration of the ethanol to ethylene. The third reaction mixture 120 then exits the adiabatic multistage reactor 106 at a second end 122 (e.g., an outlet) that is opposite the first end 118. A person skilled in the art would appreciate that the first end 118 and the second end 122 could be placed at various positions on the adiabatic multistage reactor and therefore, are not limited to their positions illustrated in FIG. 1.

As further shown in FIG. 1, the system 100 includes a first heat exchanger 124, a single stage reactor 126, a separation subsystem 128 and a second heat exchanger 130. In use, the third reaction mixture 120 is first introduced into the first heat exchanger 124 such that the temperature of the third reaction mixture 120 is decreased. The resulting cooled third reaction mixture 121 is then introduced as the input feed into the single stage reactor 126. The single stage reactor 126 includes a reactor bed 132 with one or more catalysts so that at least a portion of the ethylene in the third reaction mixture 120 is converted into higher carbon olefins (e.g., C3 to C5 olefins). In other words, when the third reaction mixture 120 contacts the reactor bed 132 of the single stage reactor 126, an output stream 134 is produced. The output stream 134 includes one or more product olefins (e.g., C3 to C5 olefins) and ethylene.

The output stream 134 can then optionally be first introduced into the condenser of the heat integration subsystem 104. Thereafter, the condensed output stream 136 can be introduced in the separation subsystem 128 that produces a first stream 138 and a second stream 140 (C3+ olefins). The first stream 138 includes ethylene, and the second stream includes C3-C5 olefins, water, and co-products (e.g., aromatic(s) and/or saturate(s)). In this illustrated system, the first stream 138 is then introduced into the second heat exchanger 130 to increase the temperature of ethylene, followed by mixing with the first portion of the ethanol 102a to form the first input stream 116. Thus, at the beginning of the process, the first input feed 116 includes only the first portion of ethanol 102a, but as the process continues, the first stream 138 (e.g., produced ethylene recycle) is combined with the first portion of the ethanol 102a upstream of the multistage reactor 106 such that the first input stream 116 then includes a combination of the ethanol 102a and the produced ethylene recycle 138.

In some aspects, the majority of the ethylene is drawn out of the system, rather than being recycled back into the system, and thus, recycled back into the multistage reactor. As such, in instances where the majority of the ethylene is drawn out of the output stream, it may be desirable to heat the first input feed, e.g., to a temperature of about 450° C., prior to introducing the ethanol portions into the multistage reactor. The heating of the ethanol can be carried out, by way of example, a furnace, electrical heater, or other heat source, that is implemented into the system.

As noted above, in some aspects, the first stream 138 can be ethylene (e.g., ethylene being the greatest concentration component of the first stream). In other aspects, it may be desirable to limit or avoid the separation of the olefins. In such aspects, the first stream can include a variety of olefin mixtures. For example, in some aspects, the first stream can include ethylene and propylene (e.g., ethylene and propylene being the greatest concentration components of the first stream). In other aspects, the first stream can include of C2-C4 olefins (e.g., C2-C4 olefins being the greatest concentration components). In yet other aspects, the first stream can include C2-C5 olefins. In some aspects, a portion of the condensed output stream can be added to the first stream prior to be separating via the separation subsystem.

In certain aspects, the one or more olefins can be generated directly from C2+ alcohols in a separate reactor using a dehydration specific catalyst and thereafter used as part of the first input feed without separation form unconverted alcohols, the water generated, or any co-products.

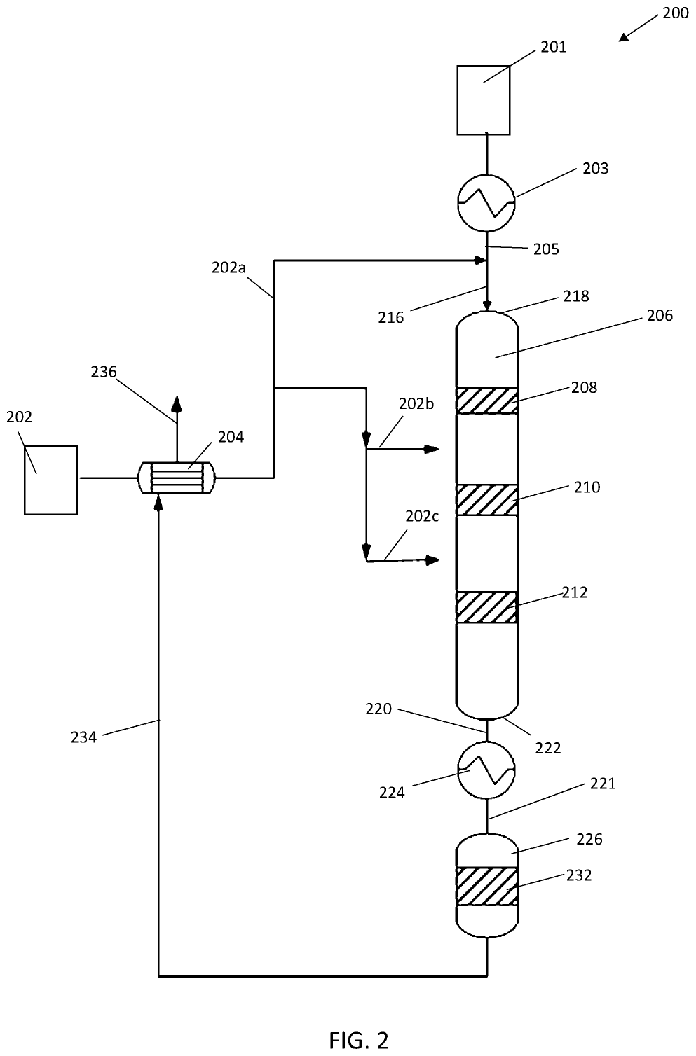

In some aspects, the reactor can have additional streams that are introduced into the reactor, in which the first input feed can also include methanol and/or dimethylether. An exemplary schematic of ethanol feed and an methanol and/or dimethylether feed system for conversion of oxygenates is illustrated in FIG. 2. For simplicity purposes only, this exemplary system 200 is discussed with respect to two streams that are combined to form the first input stream that is introduced into the adiabatic reactor. More specifically, the first stream being ethanol and the second stream being methanol and/or dimethylether. A person skilled in the art would appreciate that this exemplary system can be used for other oxygenates and olefins, and therefore is not limited to methanol, dimethylether, or ethanol.

In the illustrated system 200, the system 200 includes a source of methanol 201, an optional first heat exchanger 203, a source of ethanol 202, an optional heat integration subsystem 204, and an adiabatic multistage reactor 206. While the adiabatic multistage reactor 206 can have 2 or more reaction stages, in this illustrated example, the adiabatic multistage reactor includes three reaction stages. The first reaction stage includes a first reactor bed 208, the second reaction stage includes a second reactor bed 210, and the third reaction stage includes a third reactor bed 212. Each reactor bed 208, 210, 212 includes one or more catalysts. As will be described in more detail below, in this illustrated system, the heated methanol is provided to the adiabatic multistage reactor 206 in one portion and the heated ethanol is provided to the adiabatic multistage reactor 206 in three portions. In other aspects, additional portions of the heated methanol can be provided to the adiabatic multistage reactor, e.g., downstream of the first reactor bed. While the first heat exchanger 203 can have a variety of configurations, in this illustrated system, the first heat exchanger is configured to preheat the methanol, e.g., to a temperature from about 450° C. to 550° C. (e.g., about 450° C.). Further, while the heat integration subsystem 204 can have a variety of configurations, the second heat integration subsystem includes a preheater that is configured to preheat the ethanol, e.g., to a temperature of about 450° C. and a condenser that is configured to condense an output stream (e.g., the output stream of the single stage reactor) to a condensed output stream.

In use, methanol 201 is introduced into the preheater (not shown) and heated, and ethanol 102 is introduced into the preheater (not shown) and heated. A first portion of the heated ethanol 102a is then combined with a heated methanol stream 205 (e.g., a first stream) to generate a first input feed 216 that is then introduced into a first end 218 (e.g., a first inlet) of the adiabatic multistage reactor 206. In other aspects, the first portion of the heated ethanol 202a and the heated methanol stream 205 do not have to be introduced into the adiabatic multistage reactor 106 as a combined stream, but rather two separate streams. The first input feed 216 then contacts the first reactor bed 208 to thereby maintain a first temperature of the first reactor bed 208 within a first temperature range and to produce a first reaction mixture (not shown). The first reactor bed 208 is designed such that the heat produced from exothermic methanol conversion to dimethyl ether, methanol and dimethyl ether conversion to olefins, and olefin oligomerization expected is substantially, or completely, balanced with the heat absorbed by dehydration of the ethanol.

A second portion of the heated ethanol is introduced into the adiabatic multistage reactor 206 as a second input feed 202b. Upon exiting the first reaction stage, the first reaction mixture (not shown) is mixed with the second input feed 202b within the adiabatic multistage reactor 206 to produce a first effluent (not shown). The first effluent then contacts the second reactor bed 210 to thereby maintain a second temperature of the second reactor bed 210 within a second temperature range and to produce a second reaction mixture (not shown). The second reactor bed 210 is designed such that the heat produced from exothermic methanol conversion to dimethyl ether, methanol and dimethyl ether conversion to olefins, and olefin oligomerization expected is substantially, or completely, balanced with the heat absorbed by the dehydration of the ethanol to ethylene.

A third portion of the heated ethanol is introduced into the adiabatic multistage reactor 206 as a third input feed 202c. Upon exiting the second reaction stage, the second reaction mixture (not shown) is mixed with the second input feed 202c within the adiabatic multistage reactor 206 to produce a second effluent (not shown). The second effluent then contacts the third reactor bed 212 to thereby maintain a second temperature of the third reactor bed 212 within a third temperature range and to produce a third reaction mixture 220. The third reactor bed 212 is designed such that the heat produced from exothermic olefin oligomerization expected is substantially, or completely, balanced with the heat absorbed by dehydration of the ethanol to ethylene. The third reaction mixture 220 then exits the adiabatic multistage reactor 206 at a second end 222 (e.g., an outlet) that is opposite the first end 218. A person skilled in the art would appreciate that the first end 218 and the second end 222 could be placed at various positions on the adiabatic multistage reactor and therefore, are not limited to their positions illustrated in FIG. 2.

As further shown in FIG. 2, the system 200 includes a second heat exchanger 224 and a single stage reactor 226. In use, the third reaction mixture 220 is first introduced into the second heat exchanger 224 such that the temperature of the third reaction mixture 220 is decreased. The resulting cooled third reaction mixture 221 is then introduced as the input feed into the single stage reactor 226. The single stage reactor 226 includes a reactor bed 232 with one or more catalysts so that at least a portion of the ethylene in the third reaction mixture 220 is converted into higher carbon olefins (e.g., C3 to C5 olefins). In other words, when the third reaction mixture 220 contacts the reactor bed 232 of the single stage reactor 126, an output stream 234 is produced. The output stream 134 includes one or more product olefins (e.g., C3 to C5 olefins) and ethylene.

The output stream 234 can then optionally be first introduced into the condenser of the heat integration subsystem 204. Thereafter, the condensed output stream 236 can be introduced in a separation subsystem. While not shown, the separation subsystem can be similar to separation subsystem 128. Further, while not shown, an ethanol recycle stream, e.g., 114 in FIG. 1, can be produced in system 200 and introduced into the adiabatic reactor 206 as discussed above with respect to FIG. 1.

An exemplary process for converting one or more oxygenates to one or more olefins can include introducing a first input feed that includes ethanol, water, and methanol and/or olefin(s) into an adiabatic multistage reactor, where the temperature of the first input feed can be from about 400° C. to 480° C. In this exemplary process, ethanol can present in the first input feed from about 20-25 wt % of the of the first input feed, while methanol, ethylene, or other mixed olefins can be present in the first input feed from about 60-70 wt % of the first input feed, and the balance of the first input stream can be water. The temperature of the first reactor bed can be equal to or greater than about 350° C. and equal to or less than about 480° C. (350° C.≥first reactor bed temperature≤480° C.). Once the first reaction mixture is formed, it can be combined with a second input feed of ethanol to form a first effluent, where the another portion of ethanol is at temperature above about 300° C. The ethanol present in the first effluent is about 15-30 wt % of the first effluent. Water may be optionally added to the second input feed such that the total water in the first effluent would not exceed about 60 wt %. The temperature of the second reactor bed can be equal to or greater than about 350° C. and equal to or less than about 480° C. (350° C.≥first reactor bed temperature≤480° C.). Once the second reaction mixture is formed, it can be combined with a third input feed of ethanol to form a second effluent, where the ethanol is at temperature above about 300° C. The ethanol present in the second effluent is about 10-25 wt % of the second effluent. Water may be optionally added to the third input feed such that the total water in the second effluent would not exceed about 60 wt %. The temperature of the third reactor bed can be equal to or greater than about 350° C. and equal to or less than about 480° C. (350° C.≥first reactor bed temperature≤480° C.). Once the third reaction mixture is formed, it can then exit the adiabatic multistage reactor as an output stream of the adiabatic multistage reactor.

In instances where additional downstream reactors beds are present in the multistage reactor, the process can also include: Once the third reaction mixture is formed, it can be combined with a fourth input feed of ethanol to form a third effluent, where the ethanol is at temperature above about 300° C. The ethanol present in the second effluent is about 8-22 wt % of the third effluent. Water may be optionally added to the fourth input feed such that the total water in the third effluent would not exceed about 60 wt %. The temperature of the fourth reactor bed can be equal to or greater than about 350° C. and equal to or less than about 480° C. (350° C.≥first reactor bed temperature≤480° C.). Once the fourth reaction mixture is formed, it can exit the multistage reactor as an output steam. Optionally, the output stream can be cooled via a heat exchanger and fed as a fifth input feed into multistage reactor to a fifth reactor bed at an input temperature, e.g., from about 350° C. to 400° C. Water may be optionally added to the fifth input stream such that the total water in the fifth input feed would not exceed about 60 wt %.

Another exemplary process for converting one or more oxygenates to one or more olefins can include introducing a first input feed that includes ethanol, water, and methanol and/or olefin(s) into an adiabatic multistage reactor, where the temperature of the first input feed can be from about 400° C. to 480° C. In this exemplary process, ethanol can present in the first input feed from about 25-30 wt % of the of the first input feed, while methanol, ethylene, or other mixed olefins can be present in the first input feed from about 50-60 wt % of the first input feed, and the balance of the first input stream can be water. The temperature of the first reactor bed can be equal to or greater than about 350° C. and equal to or less than about 480° C. (350° C.≥first reactor bed temperature≤480° C.). Once the first reaction mixture is formed, it can be combined with a second input feed of ethanol to form a first effluent, where the another portion of ethanol is at temperature above 300° C. The ethanol present in the first effluent is about 20-30 wt % of the first effluent. Water may be optionally added to the second input feed such that the total water in the first effluent would not exceed about 50 wt %. The temperature of the second reactor bed can be equal to or greater than about 350° C. and equal to or less than about 480° C. (350° C.≥first reactor bed temperature≤480° C.). Once the second reaction mixture is formed, it can be combined with a third input feed of ethanol to form a second effluent, where the ethanol is at temperature above about 300° C. The ethanol present in the second effluent is about 15-25 wt % of the second effluent. Water may be optionally added to the third input feed such that the total water in the second effluent would not exceed about 50 wt %. The temperature of the third reactor bed can be equal to or greater than about 350° C. and equal to or less than about 480° C. (350° C.≥first reactor bed temperature≤480° C.). Once the third reaction mixture is formed, it can then exit the adiabatic multistage reactor as an output stream of the adiabatic multistage reactor.

In instances where additional downstream reactors beds are present in the multistage reactor, the process can also include: Once the third reaction mixture is formed, it can be combined with a fourth input feed of ethanol to form a third effluent, where the ethanol is at temperature above about 300° C. The ethanol present in the second effluent is about 15-25 wt % of the third effluent. Water may be optionally added to the fourth input feed such that the total water in the third effluent would not exceed about 50 wt %. The temperature of the fourth reactor bed can be equal to or greater than about 350° C. and equal to or less than about 480° C. (350° C.≥first reactor bed temperature≤480° C.). Once the fourth reaction mixture is formed, it can exit the multistage reactor as an output steam. Optionally, the output stream can be cooled via a heat exchanger and fed as a fifth input feed into multistage reactor to a fifth reactor bed at an input temperature, e.g., from about 350° C. to 400° C. Water may be optionally added to the fifth input stream such that the total water in the fifth input feed would not exceed about 60 wt %.

In any of the foregoing exemplary processes described above, in instances where the first input stream includes at least one or more first oxygenates and one or more first olefins, the net molar ratio of ethylene to ethanol fed across all reaction stages can be, for example, 1:1. Based on the number of reactor beds within the adiabatic multistage reactor, this molar ratio can either increase or decrease. For example, in some aspects where the multistage reactor has greater than 5 reactor beds, the net molar ratio of ethylene to ethanol fed across all reaction stages can be, for example 1:5 or 1:9.

Terminology used herein is for the purpose of describing particular embodiments and implementations only and is not intended to be limiting. For example, as used herein, the singular forms “a,” “an,” and “the” are intended to include the plural forms as well, unless the context clearly indicates otherwise.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

As used herein in the specification and claims, including as used in the examples and unless otherwise expressly specified, all numbers can be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. The word “about” or “approximately” when immediately preceding a numerical value means a range of plus or minus 10% of that value, e.g., “about 50” means 45 to 55, “about 25,000” means 22,500 to 27,500, etc. Furthermore, the phrases “less than about” a value or “greater than about” a value should be understood in view of the definition of the term “about” provided herein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes can be made to various embodiments without departing from the teachings herein. For example, the order in which various described method steps are performed may often be changed in alternative embodiments, and in other alternative embodiments, one or more method steps may be skipped altogether. Optional features of various device and system embodiments may be included in some embodiments and not in others. Therefore, the foregoing description is provided primarily for exemplary purposes and should not be interpreted to limit the scope of the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Use of the term “based on,” herein and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described herein can be directed to various combinations and subcombinations of the disclosed features and/or combinations and subcombinations of several further features disclosed herein. In addition, the logic flows depicted in the accompanying figures and/or described herein do not necessarily require the particular order shown, or sequential order, to achieve desirable results. Other implementations may be within the scope of the following claims.

1. A process for converting one or more oxygenates to one or more olefins, the process comprising:
introducing a first input feed into a first end of an adiabatic multistage reactor, the first input feed comprising one or more first oxygenates and one or more first olefins, the multistage reactor having at least a first reaction stage and a second reaction stage, wherein the first reaction stage is upstream of the second reaction stage, and the first reaction stage having a first reactor bed, and the second reaction stage having a second reactor bed;
contacting the first input feed with the first reactor bed to thereby maintain a first temperature of the first reactor bed within a first temperature range from about 300° C. to 550° C. and to produce a first reaction mixture;
introducing a second input feed into the multistage reactor downstream of the first reaction stage such that, upon exiting the first reaction stage, the first reaction mixture is mixed with the second input feed to produce a first effluent having a different composition relative to the first reaction mixture, the second input feed comprising one or more second oxygenates; and
contacting the first effluent with the second reactor bed to thereby maintain a second temperature of the second reactor bed within a second temperature range from about 300° C. to 550° C. and to produce a second reaction mixture.

2. The process of claim 1, wherein the multistage reactor has a third reaction stage, the third reaction stage having a third reactor bed, the process further comprising,
introducing a third input feed into the multistage reactor downstream of the second reaction stage such that, upon exiting the second reaction stage, the second reaction mixture is mixed with the third input feed to produce a second effluent having a different composition relative to the second reaction mixture, the third input feed comprising one or more third oxygenates; and
contacting the second effluent with the third reactor bed to thereby maintain a third temperature of the third reactor bed within a third temperature range from about 300° C. to 550° C. and to produce a third reaction mixture.

3. The process of claim 1, wherein, during each reaction stage and between reaction stages, external heat is not added to the multistage reactor.

4. The process of claim 1, wherein, during each reaction stage and between reaction stages, heat is not removed from the multistage reactor.

5. The process of claim 1, wherein at least one of the first effluent or the second effluent is not removed from the multistage reactor.

6. (canceled)

7. The process of claim 1, wherein heat is not removed from the first reaction mixture prior to being mixed with the second input feed.

8. The process of claim 1, wherein heat is not removed from the second reaction mixture prior to being removed from the multistage reactor or subsequently mixed with the third input feed.

9. (canceled)

10. The process of claim 1, wherein the one or more first oxygenates comprises a predominant first oxygenate, and the one or more first olefins comprises a predominant first olefin, wherein the predominant first oxygenate is ethanol and the predominant first olefin is ethylene.

11.-21. (canceled)

22. The process of lclaim 1, wherein the one or more first oxygenates and the one or more second oxygenates are the same.

23. (canceled)

24. The process of claim 1, wherein at least one of the one or more first oxygenates or the one or more second oxygenates comprise one or more C2+ alcohols.

25.-26. (canceled)

27. The process of claim 1, wherein the one or more first oxygenates comprise a predominate first oxygenate, wherein the predominant first oxygenate is ethanol.

28. The process of claim 1, wherein the one or more second oxygenates comprises a second predominant oxygenate, wherein the predominant second oxygenate is ethanol.

29. (canceled)

30. The process of claim 1, further comprising introducing the second reaction mixture into a single stage reactor, the single stage reactor comprising one or more catalysts, and contacting the second reaction mixture with the one or more catalysts to produce an output stream comprising one or more product olefins.

31.-49. (canceled)

50. The process of claim 1, further comprising, prior to introducing the first input stream into the adiabatic multistage reactor, heating the one or more first oxygenates.

51.-63. (canceled)

64. The process of claim 1, wherein at least one of the one or more first oxygenates or the one or more second oxygenates do not comprise methanol.

65.-66. (canceled)

67. A process for converting one or more oxygenates to one or more olefins, the process comprising:
introducing a first input feed into a first end of an adiabatic multistage reactor, the first input feed comprising one or more first oxygenates, the one or more first oxygenates comprising ethanol and at least one of methanol or dimethylether, the multistage reactor having at least a first reaction stage and a second reaction stage, wherein the first reaction stage is upstream of the second reaction stage, and the first reaction stage having a first reactor bed, and the second reaction stage having a second reactor bed;
contacting the first input feed with the first reactor bed to thereby maintain a first temperature of the first reactor bed within a first temperature range from about 300° C. to 550° C. and to produce a first reaction mixture;
introducing a second input feed into the multistage reactor downstream of the first reaction stage such that, upon exiting the first reaction stage, the first reaction mixture is mixed with the second input feed to produce a first effluent having a different composition relative to the first reaction mixture, the second input feed comprising one or more second oxygenates; and
contacting the first effluent with the second reactor bed to thereby maintain a second temperature of the second reactor bed within a second temperature range from about 300° C. to 550° C. and to produce a second reaction mixture.

68.-71. (canceled)

72. The process of claim 67, wherein the one or more second oxygenates comprises ethanol, methanol, or a combination thereof.

73.-75. (canceled)

76. A system comprising the adiabatic multistage reactor of claim 1.

77. A process for converting one or more oxygenates to one or more olefins, the process comprising:
introducing a first input feed into a first end of adiabatic multistage reactor assembly, the first input feed comprising one or more first oxygenates and at least one or more first olefins and methanol, the multistage reactor assembly having at least a first reaction stage and a second reaction stage, wherein the first reaction stage is upstream of the second reaction stage, and the first reaction stage having a first reactor bed, and the second reaction stage having a second reactor bed;
contacting the first input feed with the first reactor bed to thereby maintain a first temperature of the first reactor bed within a first temperature range from about 300° C. to 550° C. and to produce a first reaction mixture;
introducing a second input feed into the multistage reactor assembly downstream of the first reaction stage such that, upon exiting the first reaction stage, the first reaction mixture is mixed with the second input feed to produce a first effluent having a different composition relative to the first reaction mixture, the second input feed comprising one or more second oxygenates; and
contacting the first effluent with the second reactor bed to thereby maintain a second temperature of the second reactor bed within a second temperature range from about 300° C. to 550° C. and to produce a second reaction mixture.

78.-86. (canceled)

87. The process of claim 1, wherein the one or more first olefins comprise one or more recycled olefins, wherein the one or more recycled olefins comprise ethylene, propylene, butenes, pentenes, or any combination thereof.
